(19) United States
(12) Patent Application Publication
(10) Pub. No.: US 2024/0190803 A1
(43) Pub. Date: Jun. 13, 2024

(54) LOW-LEAD AND LOW-ARSENIC BETA-HYDROXYBUTYRATE SALTS AND METHODS FOR PRODUCING THE SAME

(71) Applicant: NANJING NUTRABUILDING BIO-TECH CO., LTD., Nanjing, Jiangsu (CN)

(72) Inventors: Jinjian Zhu, Nanjing (CN); Jian Zhang, Nanjing (CN); Qiru Fan, Nanjing (CN); Ronghua Yi, Nanjing (CN); Kylin Liao, Nanjing (CN)

(73) Assignee: NANJING NUTRABUILDING BIO-TECH CO., LTD., Nanjing (CN)

(21) Appl. No.: 17/768,388

(22) PCT Filed: Apr. 16, 2021
(86) PCT No.: PCT/CN2021/087769

§ 371 Date: Apr. 12, 2022

(51) Int. Cl. C07C 59/01; A61K 31/19; A61P 3/00

(52) U.S. Cl. CPC C07C 59/01; A61K 31/19; A61P 3/00; C07B 2200/07

(57) ABSTRACT

Among others, the present invention provides low-lead and low-arsenic beta-hydroxybutyrate (BHB) salts, comprising lead ranging from 10 to 50 parts per billion (ppb) and arsenic ranging from 10 to 150 ppb. The present invention also provides methods for preparing low-lead and low-arsenic beta-hydroxybutyrate (BHB) salts, which may include (a) synthesizing, (b) adsorbing (e.g., by adsorbent and solvent), (c) concentrating under reduced pressure, and (d) spray drying.

### FIELD OF THE INVENTION

This invention generally relates to the field of chemical synthesis, and more specifically relates to synthesis and preparation of low-lead and low-arsenic beta-hydroxybutyrate (BHB) salts.

### BACKGROUND OF THE INVENTION

Nutritional, or therapeutic, ketosis is the physiological state of elevated blood ketone body levels resulting from ketogenic diets, calorie restriction, therapeutic fasting, and/or supplementation with ketogenic precursors. When in ketosis, the body is essentially burning fat for its primary fuel, and begins cleaving fats into fatty acids and glycerol and transforms the fatty acids into acetyl COA molecules, which are then eventually transformed through ketogenesis into ketone bodies beta-hydroxybutyrate (beta-hydroxybutyrate or “BHB”), acetoacetate (acetylacetonate), and acetone in the liver. As such, BHB is a natural ketone body that produces by the human liver from the stored fat, which assists in breakdown of fat, fasten circulation of blood, activated the formation of new blood cells, and provides powerful energy to the brain, bone, myocardial tissues, etc.

BHB salts are pharmaceutics or supplements that contain a ketone (BHB) bound to a mineral. Released BHB not only can increase blood ketone levels, but also is able to assist entry of the ketogenic state more quickly and promote or maintain ketosis state. Furthermore, BHB can help improve endurance performance, and support better appetite management.

While Beta-hydroxybutyrate (BHB) salts have been widely commercialized as a dietary supplement, they may result in the excess intake of heavy metal ions, particularly lead and arsenic. In the current BHB salts in the market, the contents of the lead and arsenic have been neither sufficiently nor effectively controlled.

Particularly, lead and arsenic are both heavy metals. Since heavy metals generally cannot be metabolized in the body, the more they accumulate in the body, the greater damage they may cause to the body.

Lead is not a mineral required by the human body, and is potentially harmful to the human body if overdosed. This heavy metal can accumulate in the body for a long period. When the lead in the body accumulates to a certain extent, it can have toxic effects on the hematopoietic system, nervous system, kidneys, etc. Accordingly, as lead has no physiological function in the human body but harmful effects, the level of lead in human blood should be as low as possible.

Arsenic acts on the nervous system and stimulates hematopoietic organs. Although a low amount of arsenic helps the synthesis of hemoglobin and can promote the growth and development of the human body, a mass of arsenic invades the human body for a long time and has a stimulating effect on red blood cell production. Long-term exposure to abundant arsenic might cause cell poisoning and capillary poisoning. What's worse, it may also induce malignant tumors. Accordingly, conventionally produced BHB salts with insufficiently controlled lead and arsenic contents may result in the excess intake of heavy metal ions and be harmful to the human body.

To overcome these drawbacks, it is therefore desired to have improved, safer, and healthier BHB salts, and effective production methods thereof, with sufficiently low levels of lead and arsenic.

### SUMMARY OF THE INVENTION

This summary is provided to introduce a selection of concepts in a simplified form that is further described below in the Detailed Description. This summary is not intended to identify key features or essential features of the claimed subject matter, nor is it intended to be used to limit the scope of the claimed subject matter.

The present invention generally relates to low-lead, low-arsenic, and high yield beta-hydroxybutyrate (BHB) salts, and preparation methods thereof. Particularly, the preparation process according to the present invention includes an adsorption process (by particular adsorbent(s) and adsorbed solvent) for effectively adsorbing, controlling, and reducing the amounts of heavy metals including lead and arsenic. As a result, the heavy metals such as lead and arsenic can be successfully and strictly controlled through simple manufacturing process at a low cost. The prepared low-lead and low-arsenic BHB salts according to the present invention are pure, chemically stable, and also safer and healthier than the existing BHB salts in the market. The BHB salts according to the present invention may be widely used in dietary supplementary, food additives and/or pharmaceuticals, and can avoid access intake of metal heavy ions into the human body.

One aspect of this invention relates to a beta-hydroxybutyrate (BHB) salt, comprising low lead and arsenic contents, wherein the level of lead ranges from 10 to 50 parts per billion (ppb), and the level of arsenic ranges from 10 to 150 ppb. In some further embodiments, the range of lead may be 10 to 30 ppb, and/or the range of the arsenic may be 10 to 50 ppb.

In some embodiments, the BHB salt comprises a BHB metal salt. In some further embodiments, the BHB salt is formed from sodium, potassium, calcium, magnesium, or a mixture thereof (e.g., a mixture of any two or any three thereof, in any ratio).

In some embodiments, the BHB salt comprises D-BHB, DL-BHB, L-BHB form or a mixture thereof (e.g., a mixture of any two or any three thereof, in any ratio).

In some embodiments, the BHB salt has a chemical purity of at least 95% (e.g., 95.0-99.5%). Preferably, the BHB salt may have a chemical purity of at least 98%. (e.g., 98-99.5%).

In some embodiments, the BHB salt has an optical purity of at least 95% (e.g., 95.0-99.0%). Preferably, the BHB salt may have an optical purity of at least 98%. (e.g., 98-99.0%).

In some embodiments, the BHB salt is formed with a molar yield of at least 81% (e.g., 81%-95%). Preferably, the BHB salt may be formed with a molar yield of at least 88% (e.g., 88%-95%).

Another aspect of the present invention provides a composition, for promoting and/or sustaining ketosis in a mammal (e.g., human), comprising a BHB salt as described above.

In a further aspect, the prevent invention relates to a method for preparing beta-hydroxybutyrate (BHB) salt, comprising an adsorbing process for controlling and enabling low levels of lead and arsenic in the prepared BHB salt.

In some embodiments, the method comprises the steps of (a) synthesizing, (b) adsorbing, (c) concentrating under reduced pressure, and (d) spray drying.

In some further embodiments, the synthesizing step comprises adding (R)-3-hydroxybutyrate, water, and one or more metal oxides to obtain a mixture solution.

In some embodiments, the adsorbing process comprising adding adsorbent(s) and filtering solution after the adsorption. Examples of the adsorbents include but are not limited to activated carbon, normal silicone, mercaptoalkyl-functionalisedsilica, and Al₂O₃. Preferably, the adsorbent may comprise activated carbon or mercaptoalkyl-functionalisedsilica.

In some embodiments, the amount of the adsorbent is 1-50%, preferably 1-10%.

In some embodiments, the adsorbing process uses a solvent. Examples of solvents include but are not limited to water, ethanol, and methanol. For instance, the solvent is water. In some embodiments, the volume of the solvent is controlled within a suitable amount of 500 mL.

In some embodiments, the adsorbing process is operated at a temperature ranging from 20 to 60° C.

In some embodiments, the adsorbing process is operated for a time ranging from 2 to 24 hours.

In some embodiments, the concentrating under reduced pressure step comprises concentrating the solution after the adsorption process under reduced pressure. In some embodiments, the spray-dry step comprises spray-drying the concentrated solution to obtain the beta-hydroxybutyrate (BHB) salt with low lead and arsenic contents.

In some embodiments, the method effectively controls the lead and arsenic contents within safe and healthy ranges. The prepared beta-hydroxybutyrate (BHB) salt may include lead at a level ranging from 10 to 50 ppb and arsenic at a level ranging from 10 to 150 ppb. In some further embodiments, the range of lead may be 10 to 30 ppb, and/or the range of the arsenic may be 10 to 50 ppb.

In some embodiments, the prepared BHB salt is formed from sodium, potassium, calcium, magnesium, or a mixture thereof.

In some embodiments, the prepared BHB salt has a chemical purity of at least 95%, an optical purity of at least 95%, and/or a molar yield of at least 81%. In some further embodiments, the prepared BHB salt has a chemical purity of at least 98%, an optical purity of at least 98%, and/or a molar yield of at least 88%.

As used herein, the term “or” is meant to include both “and” and “or.” In other words, the term “or” may also be replaced with “and/or.”

As used herein, the singular forms “a,” “an” and “the” are intended to include the plural forms as well, unless the context clearly indicates otherwise.

### DETAILED DESCRIPTION OF THE INVENTION

Reference will now be made in detail to the preferred embodiments of the invention, examples of which are further illustrated. While the invention will be described in conjunction with the preferred embodiments, it will be understood that they are not intended to limit the invention to these embodiments. On the contrary, the invention is intended to cover alternatives, modifications, and equivalents, which may be included within the spirit and scope of the invention as defined by the claims. Furthermore, in the detailed description of the present invention, numerous specific details are set forth in order to provide a thorough understanding of the present invention. However, it will be obvious to one of ordinary skill in the art that the present invention may be practiced without these specific details. In other instances, well known methods, procedures, components, and other features have not been described in detail as not to unnecessarily obscure aspects of the present invention.

Generally speaking, various embodiments of the present invention provide for synthesis and preparation method of low-lead and low-arsenic beta-hydroxybutyrate (BHB) salts. The process steps may include: synthesizing, adsorbing, concentrating under reduced pressure and spray drying. In particular, the adsorbing process is critical for successfully and strictly controlling the amounts of heavy metals such as lead and arsenic. The BHB salt can be adsorbed by particular adsorbents (e.g., activated carbon, mercaptoalkyl-functionalisedsilica, normal silicone, or aluminum oxide (Al2O3)) and adsorbed solvent (e.g., water, ethanol, or methanol). For instance, the amount of adsorbents may be 1-50%, preferably 1-10%; and the volume of the solvent may be controlled within 500 mL.

Accordingly, the prepared BHB salt contains lead and arsenic in sufficiently low ranges, thereby achieving safer and healthier BHB salts that can be used widely and safely as a dietary supplement, food additives, etc. For instance, the level of lead may be 10-50 ppb, preferably 10-30 ppb; and/or the level of arsenic may be 10-150 ppb, preferably 10-50 ppb. In addition, the synthetic BHB salts according to the present invention have high yield (e.g., a molar yield of at least 81%, preferably 88%), and high purity (e.g., chemical purity of 95.0-99.5%, preferably 98.0-99.5%; and optical purity of 95.0-99.0%, preferably 98.0-99.0%). Furthermore, the synthesis processes according to the present invention are simple, easy to control and repeat, effective and efficient, and at a low cost.

In the present application, the BHB salt may be a BHB metal salt, e.g., formed from sodium, potassium, calcium, magnesium, or a mixture thereof. Table 1 below shows law of BHB Ca salt or Ca—Mg salt adsorption and the law of BHB.

TABLE 1

| No. | BHB salt | Adsorbent | Solvent | Temperature | Time | Lead content | Arsenic content |
|---|---|---|---|---|---|---|---|
| 1 | Ca salt | / | / | / | / | 100-150 ppb | 300-400 ppb |
| 2 | Ca—Mg salt | / | / | / | / | 80-120 ppb | 250-300 ppb |
| 3 | Ca salt | Activated carbon | Water | 20-30° C. | 2 h-24 h | 30-45 ppb | 50-150 ppb |
| 4 | Ca salt | Al₂O₃ | Water | 20-30° C. | 2 h-24 h | 50-80 ppb | 150-200 ppb |
| 5 | Ca salt | Normal silicone | Water | 20-30° C. | 2 h-24 h | 50-80 ppb | 50-150 ppb |
| 6 | Ca salt | Mercaptoalkyl- | Water | 20-30° C. | 2 h-24 h | 20-30 ppb | 20-100 ppb |
| | | functionalisedsilica | | | | | |
| 7 | Ca salt | Activated carbon | Methanol | 20-30° C. | 2 h-24 h | 80-100 ppb | 300-400 ppb |
| 8 | Ca salt | Activated carbon | Ethanol | 20-30° C. | 2 h-24 h | 80-100 ppb | 300-400 ppb |
| 9 | Ca salt | Activated carbon | Water | 50-60° C. | 2 h-24 h | 30-50 ppb | 100-150 ppb |
| 10 | Ca salt | Activated carbon | Water | 20-30° C. | 2 h-24 h | 30-50 ppb | 100-150 ppb |
| 11 | Ca—Mg salt | Activated carbon | Water | 20-30° C. | 2 h-24 h | 10-40 ppb | 10-100 ppb |
| 12 | Ca—Mg salt | Al₂O₃ | Water | 20-30° C. | 2 h-24 h | 50-80 ppb | 150-200 ppb |
| 13 | Ca—Mg salt | Normal silicone | Water | 20-30° C. | 2 h-24 h | 50-80 ppb | 100-150 ppb |
| 14 | Ca—Mg salt | Mercaptoalkyl- | Water | 20-30° C. | 2 h-24 h | 10-30 ppb | 10-50 ppb |
| | | functionalisedsilica | | | | | |

In Table 1, Nos. 1-2 are non-adsorption samples. For Nos. 3-14, adsorbents are added in 1-10%. As shown in Table 1, use of adsorbents (e.g., activated carbon, mercaptoalkyl-functionalisedsilica, normal silicone, and Al₂O₃) and solvents (e.g., water, ethanol, and methanol) significantly and effectively decrease the lead and arsenic contents in the BHB salts. In particular, the activated carbon and mercaptoalkyl-functionalisedsilica show great adsorption effects on lead and arsenic. The adsorption effect in aqueous solution is also shown to be better than that in alcohol solution.

The adsorption law of potassium and sodium salts is also found to be similar to that of Ca and Ca—Mg salts, as described above.

Further, the following examples are illustrative of select embodiments of the present invention and are not meant to limit the scope of the invention, including, e.g., (R)-3-hydroxybutanoic acid sodium salt, (R)-3-hydroxybutanoic acid potassium salt, (R)-3-hydroxybutanoic acid calcium salt, (R)-3-hydroxybutanoic acid magnesium salt, (R)-3-hydroxybutanoic acid calcium and magnesium salt and (R)-3-hydroxybutyric acid.

### Preparation of (R)-3-Hydroxybutanoic Acid Sodium Salt

### Example 1

Add 100 g of methyl (R)-3-hydroxybutyrate and 500 ml of water into the reaction bottle, and then add 34.2 g of sodium hydroxide in batches at 10-20° C. Stir the mixture solution at 70-80° C. for 2 h. The endpoint of reaction was detected by HPLC. After cooling to 20-30° C., add 5 g of activated carbon and stir the mixture solution for 2 h. Filter the mixture solution and then concentrate it to about 200 mL under reduced pressure. At last, spray-dry it to obtain 98.2 g of a white solid (R)-3-hydroxybutanoic acid sodium salt with the lead content of 30 ppb and arsenic content of 120 ppb, of which the molar yield was 92%, the chemical purity was 99.1% and the optical purity was 99.0%.

### Example 2

Add 100 g of methyl (R)-3-hydroxybutyrate and 500 ml of water into the reaction bottle, and then add 44.9 g of sodium carbonate in batches at 10-20° C. Stir the mixture solution at 70-80° C. for 2 h. The endpoint of reaction was detected by HPLC. After cooling to 20-30° C., add 5 g of activated carbon and stir the mixture solution for 2 h. Filter the mixture solution and then concentrate it to about 200 mL under reduced pressure. At last, spray-dry it to obtain 98.2 g of a white solid (R)-3-hydroxybutanoic acid sodium salt with the lead content of 25 ppb and arsenic content of 110 ppb, of which the molar yield was 92%, the chemical purity was 99.1% and the optical purity was 99.0%.

### Preparation of (R)-3-Hydroxybutanoic Acid Potassium Salt

### Example 3

Add 100 g of methyl (R)-3-hydroxybutyrate and 500 ml of water into the reaction bottle, and then add 47.5 g (after conversion by content) of potassium hydroxide in batches at 10-20° C. Stir the mixture solution at 70-80° C. for 2 h. The endpoint of reaction was detected by HPLC. After cooling to 20-30° C., add 5% activated carbon and stir the mixture solution for 2 h. Filter the mixture solution and then concentrate it to about 200 mL under reduced pressure. At last, spray-dry it to obtain 108 g of a white solid (R)-3-hydroxybutanoic acid potassium salt with the lead content of 32 ppb and arsenic content of 100 ppb, of which the molar yield was 90%, the chemical purity was 99.1% and the optical purity was 99.0%.

### Example 4

Add 100 g of methyl (R)-3-hydroxybutyrate and 500 ml of water into the reaction bottle, and then add 58.5 g of potassium carbonate in batches at 10-20° C. Stir the mixture solution at 70-80° C. for 2 h. The endpoint of reaction was detected by HPLC. After cooling to 20-30° C., add 5% activated carbon and stir the mixture solution for 2 h. Filter the mixture solution and then concentrate it to about 200 mL under reduced pressure. At last, spray-dry it to obtain 109 g of a white solid (R)-3-hydroxybutanoic acid potassium salt with the lead content of 30 ppb and arsenic content of 110 ppb, of which the molar yield was 90%, the chemical purity was 99.1% and the optical purity was 99.0%.

### Preparation Method of (R)-3-Hydroxybutanoic Acid Calcium Salt

### Example 5

Add 100 g of methyl (R)-3-hydroxybutyrate and 500 ml of water into the reaction bottle, and then add 25 g of calcium oxide (lead content: 500 ppb; arsenic: 1500 ppb) in batches at 10-20° C. Stir the mixture solution at 90-100° C. for 6 h. The endpoint of reaction was detected by HPLC. After cooling to 20-30° C., add 5 g activated carbon and stir the mixture solution for 2 h. Filter the mixture solution and then concentrate it to about 200 mL under reduced pressure. At last, spray-dry it to obtain 96.7 g of a white solid (R)-3-hydroxybutanoic acid calcium salt with the lead content of 30 ppb and arsenic content of 120 ppb, of which the molar yield was 93%, the chemical purity was 99.1% and the optical purity was 99.0%.

### Example 6

Add 100 g of methyl (R)-3-hydroxybutyrate and 500 ml of water into the reaction bottle, and add then 34.2 g of sodium hydroxide in batches at 10-20° C. Stir the mixture solution at 70-80° C. for 2 h. The endpoint of reaction was detected by HPLC. After cooling to room temperature, pass the mixture solution through a cation exchange resin column to obtain (R)-3-hydroxybutyric acid aqueous solution. Add 25 g of calcium oxide (lead content: 500 ppb; arsenic: 1500 ppb) and stir it at 80-90° ° C. for 2 h. After cooling to 20-30° C., add 5 g activated charcoal and stir the mixture solution for 2 h. Filter the mixture solution and then concentrate it to about 200 ml under reduced pressure. At last, spray-dry it to obtain 94.6 g of a white solid (R)-3-hydroxybutanoic acid calcium salt with the lead content of 32 ppb and arsenic content of 130 ppb, of which the molar yield was 91%, the chemical purity was 99.2% and the optical purity was 98.8%.

### Example 7

Add 100 g of methyl (R)-3-hydroxybutyrate and 500 ml of water into the reaction bottle, and then add 34.2 g of sodium hydroxide in batches at 10-20° C. Stir the mixture solution at 70-80° C. for 2 h. The endpoint of reaction was detected by HPLC. Concentrate the mixture solution to about 150 ml and add 300 ml of ethanol. Adjust pH 1-2 with 85.8 g of concentrated hydrochloric acid. Filtered the mixture and concentrated it under reduced pressure to solvent-free distillation. After cooling to room temperature, add 200 ml of ethanol, stir by filtering, and concentrate the filtrate to obtain (R)-3-hydroxybutyric acid oil. Add 500 ml and 25 g of calcium oxide (lead content: 500 ppb; arsenic: 1500 ppb) and stir it at 80-85° C. for 2 h. After cooling to 20-30° C., add 5 g activated carbon and stir the mixture solution for 2 h. Filter the mixture solution and then concentrate it to about 200 mL under reduced pressure. At last, spray-dry it to obtain 94.6 g of a white solid (R)-3-hydroxybutanoic acid calcium salt with the lead content of 31 ppb and arsenic content of 125 ppb, of which the molar yield was 91%, the chemical purity was 99.0% and the optical purity was 98.8%.

### Preparation of (R)-3-Hydroxybutanoic Acid Magnesium Salt

### Example 8

Add 100 g of methyl (R)-3-hydroxybutyrate and 500 mL of water into the reaction bottle, and then add 17.2 g of magnesium oxide (lead content: 100 ppb; arsenic: 50 ppb). Stir the mixture solution at 90-100° C. for 6 h. The endpoint of reaction was detected by HPLC. After cooling to room temperature, filter the mixture solution and then concentrate it to about 200 mL under reduced pressure. At last, spray-dry it to obtain 92.8 g of a white solid (R)-3-hydroxybutanoic acid magnesium salt with the lead content of 20 ppb and arsenic content of 10 ppb, of which the molar yield was 95%, the chemical purity was 99.5% and the optical purity was 99.0%.

### Example 9

Add 100 g of methyl (R)-3-hydroxybutyrate and 500 ml of water into the reaction bottle, and then add 34.2 g of sodium hydroxide in batches at 10-20° C. Stir the mixture solution at 70-80° C. for 2 h. The endpoint of reaction was detected by HPLC. After cooling to room temperature, pass the mixture solution through a cation exchange resin column to obtain (R)-3-hydroxybutyric acid aqueous solution. Add 17.2 g of magnesium oxide (lead content: 100 ppb; arsenic: 50 ppb) and stir it at 80-90° C. for 2 h. After cooling to room temperature, filter the mixture solution and then concentrate it to about 200 mL under reduced pressure. At last, spray-dry it to obtain 87.9 g of a white solid (R)-3-hydroxybutanoic acid magnesium salt with the lead content of 20 ppb and arsenic content of 10 ppb, of which the molar yield was 90%, the chemical purity was 99.3% and the optical purity was 98.9%.

### Example 10

Add 100 g of methyl (R)-3-hydroxybutyrate and 500 mL of water into the reaction bottle, and add then 34.2 g of sodium hydroxide in batches at 10-20° C. Stir the mixture solution at 70-80° C. for 2 h. The endpoint of reaction was detected by HPLC. Concentrate the mixture solution to about 150 mL and add 300 ml of ethanol. Adjust pH 1-2 with 85.8 g of concentrated hydrochloric acid. Filtered the mixture and concentrated it under reduced pressure to solvent-free distillation. After cooling to room temperature, add 200 ml of ethanol, stir by filtering, and concentrate the filtrate to obtain (R)-3-hydroxybutyric acid oil. Add 500 mL and 17.2 g of magnesium oxide (lead content: 100 ppb; arsenic: 50 ppb) and stir it at 80-90° C. for 2 h. After cooling to room temperature, filter the mixture solution and then concentrate it to about 200 ml under reduced pressure. At last, spray-dry it to obtain 87.9 g of a white solid (R)-3-hydroxybutanoic acid magnesium salt with the lead content of 10 ppb and arsenic content of 10 ppb, of which the molar yield was 90%, the chemical purity was 99.4% and the optical purity was 98.8%.

### Preparation of (R)-3-Hydroxybutanoic Acid Calcium and Magnesium Salt (w/w=1:2)

### Example 11

Add 100 g of methyl (R)-3-hydroxybutyrate and 500 mL of water into the reaction bottle, and then add 8.0 g of calcium oxide (lead content: 500 ppb; arsenic: 1500 ppb) and 11.7 g of magnesium oxide (lead content: 100 ppb; arsenic: 50 ppb). Stir the mixture solution at 90-100° C. for 6 h. The endpoint of reaction was detected by HPLC. After cooling to 20-30° C., add 5 g activated carbon and stir the mixture solution for 2 h. Filter the mixture solution and then concentrate it to about 200 mL under reduced pressure. At last, spray-dry it to obtain 95.5 g of a white solid (R)-3-hydroxybutanoic acid magnesium salt (w/w=1:2) with the lead content of 20 ppb and arsenic content of 77 ppb, of which the molar yield was 95%, the chemical purity was 99.2% and the optical purity was 99.0%.

### Example 12

Add 100 g of methyl (R)-3-hydroxybutyrate and 500 ml of water into the reaction bottle, and add then 34.2 g of sodium hydroxide in batches at 10-20° C. Stir the mixture solution at 70-80° C. for 2 h. The endpoint of reaction was detected by HPLC. After cooling to room temperature, pass the mixture solution through a cation exchange resin column to obtain (R)-3-hydroxybutyric acid aqueous solution. Add 8.0 g of calcium oxide (lead content: 500 ppb; arsenic: 1500 ppb) and 11.7 g of magnesium oxide (lead content: 100 ppb; arsenic: 50 ppb) and stir it at 80-90° C. for 2 h. After cooling to 20-30° C., add 5 g activated carbon and stir the mixture solution for 2 h. Filter the mixture solution and then concentrate it to about 200 mL under reduced pressure. At last, spray-dry it to obtain 91.1 g of a white solid (R)-3-hydroxybutanoic acid magnesium salt (w/w=1:2) with the lead content of 10 ppb and arsenic content of 10 ppb, of which the molar yield was 91%, the chemical purity was 99.5% and the optical purity was 98.9%.

### Example 13

Add 100 g of methyl (R)-3-hydroxybutyrate and 500 ml of water into the reaction bottle, and then add 34.2 g of sodium hydroxide in batches at 10-20° C. Stir the mixture solution at 70-80° C. for 2 h. The endpoint of reaction was detected by HPLC. Concentrate the mixture solution to about 150 ml and add 300 ml of ethanol. Adjust pH 1-2 with 85.8 g of concentrated hydrochloric acid. Filtered the mixture and concentrated it under reduced pressure to solvent-free distillation. After cooling to room temperature, add 200 ml of ethanol, stir by filtering, and concentrate the filtrate to obtain (R)-3-hydroxybutyric acid oil. Add 500 mL and 18.0 g of calcium oxide (lead content: 500 ppb; arsenic: 1500 ppb) and 11.7 g of magnesium oxide (lead content: 100 ppb; arsenic: 50 ppb) and stir it at 80-90° C. for 2 h. After cooling to 20-30° ° C., add 5 g activated carbon and stir the mixture solution for 2 h. Filter the mixture solution and then concentrate it to about 200 mL under reduced pressure. At last, spray-dry it to obtain 91.1 g of a white solid (R)-3-hydroxybutanoic acid magnesium salt (w/w=1:2) with the lead content of 32 ppb and arsenic content of 100 ppb, of which the molar yield was 91%, the chemical purity was 99.5% and the optical purity was 98.8%.

### Preparation of (R)-3-Hydroxybutyric Acid (50%)

### Example 14

The 50% mass concentration aqueous solution or adsorbed reaction solution (usually 20-30% mass concentration) of (R)-3-hydroxybutanoic acid sodium salt, (R)-3-hydroxybutanoic acid potassium salt, (R)-3-hydroxybutanoic acid calcium salt, (R)-3-hydroxybutanoic acid magnesium salt, or (R)-3-hydroxybutanoic acid calcium and magnesium salt (w/w=1:2) through a cation exchange resin column to collect the part product, and then concentrate it under reduced pressure to 50% mass concentration of (R)-3-hydroxybutyric acid with the lead content of 20-40 ppb and arsenic content of 10-100 ppb, of which the molar yield was 88-95%, the chemical purity was 99.0-99.5%, the optical purity was 98.0-99.0%.

### Example 15

Acidize the 50% mass concentration aqueous solution or adsorbed reaction solution (usually 20-30% mass concentration) of (R)-3-hydroxybutanoic acid sodium salt, (R)-3-hydroxybutanoic acid potassium salt, (R)-3-hydroxybutanoic acid calcium salt, (R)-3-hydroxybutanoic acid magnesium salt, or (R)-3-hydroxybutanoic acid calcium and magnesium salt (w/w=1:2) with equal molar amount of concentrated hydrochloric acid, then concentrate it under reduced pressure and add alcohol (generally 1-3 times the mass of the raw material). After stirring, filter the mixture, concentrate the filtrate to dryness under reduced pressure, and add the water to obtain (R)-3-hydroxybutyric acid (50%) with the lead content of 20-40 ppb and the arsenic content of 10-100 ppb, of which the molar yield was 90-95%, the chemical purity was 98.0-99.0%, the optical purity is 98.0-99.0%.

### Example 16

Acidize the 50% mass concentration aqueous solution or the adsorbed reaction solution (usually 20-30% mass concentration) of (R)-3-hydroxybutanoic acid calcium salt is acidified with 0.5 times the molar amount of 50% sulfuric acid. Filter the mixture and concentrate the filtrate under reduced pressure to obtain (R)-3-hydroxybutyric acid (50%) with the lead content of 20-40 ppb and the arsenic content of 10-100 ppb, of which the molar yield was 90-95%, the chemical purity was 98.0-99.0%, the optical purity is 98.50-99.0%.

Although specific embodiments and examples of this invention have been illustrated herein, it will be appreciated by those skilled in the art that any modifications and variations can be made without departing from the spirit of the invention. The examples and illustrations above are not intended to limit the scope of this invention. Any combination of embodiments of this invention, along with any obvious their extension or analogs, are within the scope of this invention. Further, it is intended that this invention encompass any arrangement, which is calculated to achieve that same purpose, and all such variations and modifications as fall within the scope of the appended claims.

All the features disclosed in this specification (including any accompanying claims, abstract and drawings) may be replaced by alternative features serving the same, equivalent or similar purpose, unless expressly stated otherwise. Thus, unless expressly stated otherwise, each feature disclosed is one example of a generic series of equivalent or similar features.

### OTHER EMBODIMENTS

It is to be understood that while the invention has been described in conjunction with the detailed description thereof and accompanying figures, the foregoing description and accompanying figures are only intended to illustrate, and not limit the scope of the invention, which is defined by the scope of the appended claims. Other aspects, advantages, and modifications are within the scope of the following claims. All publications referenced herein are incorporated by reference in their entireties.

1. A beta-hydroxybutyrate (BHB) salt, comprising low lead and arsenic contents, wherein the level of lead ranges from 10 to 50 parts per billion (ppb), and the level of arsenic ranges from 10 to 150 ppb.

2. The beta-hydroxybutyrate (BHB) salt of claim 1, wherein the level of lead ranges from 10 to 30 ppb, and the arsenic content ranges from 10-50 ppb.

3. The beta-hydroxybutyrate (BHB) salt of claim 1, wherein the BHB salt comprises a BHB metal salt.

4. The beta-hydroxybutyrate (BHB) salt of claim 3, wherein the BHB salt is formed from sodium, potassium, calcium, magnesium, or a mixture thereof.

5. The beta-hydroxybutyrate (BHB) salt of claim 1, wherein the BHB salt comprises D-BHB, DL-BHB, L-BHB form or a mixture thereof.

6. The beta-hydroxybutyrate (BHB) salt of claim 1, wherein the BHB salt has a chemical purity of at least 90%.

7. The beta-hydroxybutyrate (BHB) salt of claim 6, wherein the BHB salt has a chemical purity of at least 95%, an optical purity of at least 95%, or is formed with a molar yield of at least 81%.

8. The beta-hydroxybutyrate (BHB) salt of claim 7, wherein the BHB salt has a chemical purity of at least 98%, an optical purity of at least 98%, or is formed with a molar yield of at least 88%.

9. A composition for promoting and/or sustaining ketosis in a mammal, comprising a BHB salt of claim 1.

10. A method for preparing beta-hydroxybutyrate (BHB) salt, comprising an adsorbing process for controlling and enabling low levels of lead and arsenic in the prepared BHB salt.

11. The method of claim 10, comprising the steps of (a) synthesizing, (b) adsorbing, (c) concentrating under reduced pressure, and (d) spray drying.

12. The method of claim 11, wherein the synthesizing step comprises adding (R)-3-hydroxybutyrate, water, and one or more metal oxides to obtain a mixture solution.

13. The method of claim 10, wherein the adsorbing process comprising adding an adsorbent and filtering solution after the adsorption, wherein the adsorbent comprises activated carbon, normal silicone, mercaptoalkyl-functionalisedsilica, or Al2O3.

14. The method of claim 13, wherein the adsorbent comprises activated carbon or mercaptoalkyl-functionalisedsilica.

15. The method of claim 13, wherein the amount of the adsorbent ranges from 1 to 50%.

16. The method of claim 15, wherein the amount of the adsorbent ranges from 1 to 10%.

17. The method of claim 10, wherein the adsorbing process uses a solvent comprising water, ethanol, or methanol.

18. The method of claim 17, wherein the solvent in the adsorbing process is water.

19. The method of claim 17, wherein volume of the solvents is controlled within an amount of 500 mL.

20. The method of claim 10, wherein adsorbing process is operated at a temperature ranging from 20 to 60° C.

21. The method of claim 20, wherein the adsorbing process is operated for a time ranging from 2 to 24 hours.

22. The method of claim 11, wherein concentrating under reduced pressure step comprises concentrating the solution after the adsorption process under reduced pressure; and the spray-dry step comprises spray-drying the concentrated solution to obtain the beta-hydroxybutyrate (BHB) salt with low lead and arsenic contents.

23. The method of claim 10, wherein the method effectively controls the lead and arsenic contents within safe and healthy ranges, and the prepared beta-hydroxybutyrate (BHB) salt comprises lead at a level ranging from 10 to 50 ppb and arsenic at a level ranging from 10 to 150 ppb.

24. The method of claim 23, wherein the BHB salt comprises lead at a level ranging from 10 to 30 ppb and arsenic at a level ranging from 10-50 ppb.

25. The method of claim 23, wherein the prepared BHB salt is formed from sodium, potassium, calcium, magnesium, or a mixture thereof.

26. The method of claim 23, wherein the prepared BHB salt comprises D-BHB, DL-BHB, L-BHB form, or a mixture thereof.

27. The method of claim 23, wherein the prepared BHB salt has a chemical purity of at least 95%, an optical purity of at least 95%, or a molar yield of at least 81%.

28. The method of claim 27, wherein the prepared BHB salt has a chemical purity of at least 98%, an optical purity of at least 98%, or a molar yield of at least 88%.
